(19) United States
(12) Patent Application Publication
(10) Pub. No.: US 2010/0076542 A1
(43) Pub. Date: Mar. 25, 2010

(54) COATED EXPANDABLE SYSTEM

(75) Inventor: Michael Orlowski, Bonn (DE)

Correspondence Address: Gregory A. Nelson;Novak Druce & Quigg LLP, 525 Okeechobee Blvd, Suite 1500, West Palm Beach, FL, 33401, US

(73) Assignee: EUROCOR GMBH, Bonn (DE)

(21) Appl. No.: 12/527,741

(22) PCT Filed: Feb. 20, 2008
(86) PCT No.: PCT/DE2008/000301

§ 371 Date: Nov. 19, 2009

Related U.S. Application Data
(60) Provisional application No. 60/903,298, filed on Feb. 26, 2007.

(30) Foreign Application Priority Data
Feb. 21, 2007 (DE) 10 2007 008 479.1

(51) Int. Cl. A61F 2/84; B05D 7/00

(52) U.S. Cl. 623/1.12; 427/2.25; 427/2.3

(57) ABSTRACT

The present invention relates to an expandable system consisting of a catheter balloon and a crimped stent. The system can include a rapid release kinetic of one active agent with a slow release kinetic of a second active agent, due to the fact that the catheter balloon is coated with a first active agent adapted for rapid release and the stent is coated with a second agent adapted for slower release. The catheter balloon can be coated with a cytotoxic amount of a first active agent and the stent can be coated with a cytostatic amount of a second active agent.

The present invention relates to an expandable system consisting of a catheter balloon and a crimped stent, wherein said system combines a rapid release kinetic of one active agent with a slow release kinetic of a second active agent, due to the fact that the catheter balloon is coated with a first active agent adapted for rapid release and the stent is coated with a second agent adapted for slower release.

Nowadays, implantation of stents has become a well-established surgical intervention for the treatment of stenoses. In this context, a frequently occurring complication is so-called restenosis, i.e. reocclusion of the vessel. There's no exact definition of the term restenosis to be found in literature. The most frequently used morphological definition of restenosis defines restenosis as a reduction of the vessel diameter to less than 50% of the normal value subsequent to successful PTA (percutaneous transluminal angioplasty). Said definition describes an empirically determined value and its hemodynamic meaning and association with clinical symptoms lack scientific background. In practice, clinical deterioration in a patient is often considered a sign for the occurrence of restenosis in the previously treated vessel section.

Restenosis following stent implantation is one of the major causes for further hospitalization. Vessel traumas induced during stent implantation cause inflammatory reactions which play a decisive role in the healing process during the first seven days. The processes occurring in this context are among others associated with the release of growth factors, by means of which an increased proliferation of the smooth muscle cells is induced and thus a restenosis, a reocclusion of the vessel due to uncontrolled growth, is caused shortly thereafter.

The present invention is based on the observation that the extent and occurrence of restenosis is determined especially during the first week after stent implantation and that it is thus of particular importance to treat inflammations as well as possible during the first approximately 7 days following a successful stent implantation.

It is the object of the present invention to provide a system capable of supplying the whole surface of the stenosed tissue with high concentrations of an active agent directly after dilatation and of providing a low supply of active agent some time after the dilatation.

Said objective is resolved by the technical teaching of the independent claims. Further advantageous embodiments of the invention result from the dependent claims, the description and the examples.

The present invention discloses an expandable system consisting of a catheter balloon and an attached, crimped stent, wherein the catheter balloon is provided with an active agent having different release kinetics than the one provided on the stent.

The term “different release kinetics” refers in particular to the release rates of the two active agents.

According to the invention, the whole surface of the catheter balloon is preferably coated with an active agent. The term “active agent” refers in particular to the antiproliferative, antiinflammatory, antiangiogenic, cytostatic, cytotoxic, antithrombotic and/or antirestenotic agents mentioned further below.

The term “coating of the whole surface” means that basically the entire length of the catheter balloon is coated, which length may exceed the length of the stent and thus the catheter balloon is capable of supplying all surfaces of the obstructed vessel segment with the active agent. In contrast to conventional systems with coated stents, the active agent can only be delivered from the stent to the directly adjoining tissue. In the inventive system, the active agent is delivered from the stent and additionally from the catheter balloon into the interspaces between the stent struts when in expanded state.

According to the invention, a different active agent to the one provided on the stent may be provided on the catheter balloon, wherein, however, it is also possible that the same active agent is used both on the catheter balloon and on the stent, but in different concentrations.

Thus, the terms “a first active agent” and “a second active agent” refer to at least two different active agents or to the same active agent in two different concentrations. At least two active agents means that there may also be two or more active agents provided on the catheter balloon and one or more active agents on the stent, or two or more active agents may be provided on the stent and one or more active agents on the catheter balloon.

Thus, the expandable system is preferably provided with a combination of two active agents having different release kinetics or only one active agent in two different concentrations and with different release kinetics.

Furthermore, it is preferred according to the invention that the at least one active agent of the catheter balloon can be delivered or respectively released or respectively transferred to the vessel wall more quickly than the at least one active agent on the stent. The release rate of the at least one active agent on the stent should therefore be slower than the release rate of the at least one active agent on the catheter balloon.

It is preferred that the release rate of the at least one active agent on the catheter balloon is such that at least 10% by weight of the amount of the active agent provided on the catheter balloon are released within the period of one minute, preferably within 45 seconds, more preferably within 30 seconds and most preferably within 20 seconds, wherein the term release preferably is intended to mean the transfer to the vessel wall.

If the release kinetics are examined over a period of 30 seconds, preferably 5% by weight, more preferably 10% by weight, even more preferably 15% by weight and most preferably 20% by weight of the active agent on the catheter balloon are released at the moment of dilatation, wherein the term release is preferably intended to mean the transfer to the vessel wall.

Furthermore, it is preferred that, with respect to the stent, a 5 times higher, preferably a 10 times higher, more preferably a 20 times higher and most preferably a 30 times higher amount in mol of the active agent is applied onto the catheter balloon.

Moreover, it is preferred that a cytotoxic amount of an active agent is provided on the catheter balloon and that a cytostatic amount of an active agent is provided on the stent.

Thus, it is guaranteed that directly after dilatation a large amount of active agent is transferred to the vessel wall, by means of which the cell growth, especially of smooth muscle cells being primarily responsible for restenosis is stopped at first and then the cytostatic amount of an active agent provided on the stent leads to controlled ingrowth of the stent into the vessel wall, as the cells are not killed but their growth is reduced.

Thus, the inventive expandable system provides a combination consisting of catheter balloon and stent, which system is capable of providing a combination therapy with two active agents or with one active agent in different concentrations.

Additionally, the inventive system can be provided in two versions, i.e. in one embodiment including a biostable stent and in another embodiment including a bioresorbable stent.

In the embodiment with coated catheter balloon and coated non-bioresorbable, i.e. biostable stent crimped thereon, a stent made of conventional, preferably metallic, materials is used.

The term “non-bioresorbable” refers to the fact that the stent is a permanent implant which is not, or alternatively only very slowly, degraded under physiological conditions. For example, such stents are made of medical steel, titanium, chromium, vanadium, tungsten, molybdenum, gold, nitinol, magnesium, zinc, alloys of the aforementioned metals as well as ceramics or biostable polymers.

In the embodiment with coated catheter balloon and bioresorbable, i.e. biodegradable, stent crimped thereon, a stent made of a bioresorbable metal alloy or a biodegradable polymer is used, wherein said stent may be coated with a layer containing the pure active agent and/or one or more active agents can be inserted or incorporated into the biodegradable material itself and/or the bioresorbable stent may be coated with a biodegradable coating containing one or more active agents.

Coated Catheter Balloon with Coated Biostable Stent

This version provides an ideal system to keep a strongly obstructed body passage, such as biliary tracts, esophagus, urinary tracts, pancreas, renal tracts, pulmonary tracts, trachea, small intestine and large intestine and especially blood vessels open by using a permanent stent, which is preferably coated with a cytostatic dose of an active agent.

The catheter balloon according to this version is coated with a layer containing the pure active agent or with a carrier containing an active agent and during dilatation both the stent is placed and an active agent is applied over an area covering at least the entire length of the stent and advantageously over an area exceeding said length, which active agent leads to controlled ingrowth and prevents the stent from being overgrown by mainly smooth muscle cells. The active agents mentioned below and especially paclitaxel and/or rapamycin may be used as active agent or mixture of active agents.

Preferably, the catheter balloon is coated such with active agent with or without a carrier system that the coating of the balloon extends beyond both ends of the stent and preferably such that it extends beyond the respective ends of the stent to an extent of about 10 to 20% of the total length of the stent. During dilatation, the active agent is therefore also transferred to the vessel area at both ends of the stent which is not covered by the stent and active agent is transferred to the whole surface of the vessel wall situated between the expanding or respectively expanded struts or the stent.

Said embodiment has the advantage that the stent surface is provided with an active agent which, however, preferably does not cause cell death of the smooth muscle cells but only inhibits their growth and thus no cells in immediate contact with the stent surface are killed. However, in the interspaces between the struts and also at the ends of the stent, or respectively in the two areas of the vessel wall extending beyond the ends of the stent, a sufficient amount of active agent is applied so that especially a too rapid overgrowth of the stent beginning in the interspaces of the stent struts and continuing inside the stent and leading to the occurrence of in-stent restenosis is confined or respectively reduced to a controlled level.

According to this version, the active agent is applied directly where it is required while in a stent coated with active agent said active agent is only released from the surface of the stent but not from the interspaces between the struts of the stent or at the ends of the stent or respectively the area extending beyond the stent and is additionally delivered explicitly to the adjoining tissue which is not to be inhibited or killed.

Consequently, the catheter balloon is preferably coated with the active agent, with or without carrier, and afterwards a coated stent is crimped onto the balloon. Suitable carrier systems for the active agent provided on the catheter balloon and on the stent are described in greater detail further below.

The embodiment including a bioresorbable stent will probably gain importance in future, as said embodiment does not represent a permanent implant. Said embodiment uses biodegradable, i.e. bioresorbable stents. Such stents which are degradable under physiological conditions are completely decomposed in the patient's body within a period of a few weeks to up to one or two years.

Biodegradable stents consist of metals such as magnesium, calcium or zinc, or of organic compounds such as polyhydroxy butyrate, chitosan or collagen.

A bioresorbable metal stent consisting mainly of magnesium is disclosed in European patent EP 1 419 793 B1. The German published application describes stents made of magnesium alloys and zinc alloys. Bioresorbable stents made of magnesium, calcium, titanium, zirconium, niobium, tantalum, zinc or silicon or of alloys or mixtures of the aforementioned substances are disclosed in the German published patent application DE 198 56 983 A1. Explicit examples regarding stents made of a zinc-calcium alloy are disclosed.

Other bioresorbable metal stents made of magnesium, titanium, zirconium, niobium, tantalum, zinc and/or silicon as component A and of lithium, sodium, potassium, calcium, manganese and/or iron as component B are described in European patent application EP 0 966 979 A2. Explicit examples regarding stents made of a zinc-titanium alloy including titanium to a quantity of from 0.1 to 1% by weight and a zinc calcium alloy having a weight ratio between zinc and calcium of 21:1 are disclosed therein.

A biodegradable stent consisting of the organic compound polyhydroxy butyrate

(PHB) and other polyhydroxy alkanoates is disclosed in US patents U.S. Pat. No. 6,548,569 B1, U.S. Pat. No. 5,935,506, U.S. Pat. No. 6,623,749 B2, U.S. Pat. No. 6,838,493 B2 and U.S. Pat. No. 6,867,247 B2.

Furthermore, U.S. Pat. No. 6,245,103 B1 mentions polydioxanones, polycaprolactones, polygluconates, copolymers of polylactic acid and polyethylene oxide, modified cellulose, collagen, poly(hydroxy butyrates), polyanhydrides, polyphosphoesters and polyamino acids as additional suitable biodegradable stent materials.

U.S. Pat. No. 6,991,647 B2 further mentions polyglcolic acid, polylactides, polyphosphate esters and poly-ε-caprolactone as potential biodegradable organic polymers.

Generally, biodegradable stents may be made of the following substances or mixtures of the following substances: polyvalerolactone, poly-ε-decalactone, polylactides, polyglycolides, copolymers of the polylactides and polyglycolides, poly-ε-caprolactone, polyhydroxybutyric acid, polyhydroxybutyrates, polyhydroxyvalerates, polyhydroxybutyrate-co-valerate, poly(1,4-dioxan-2,3-one), poly(1,3-dioxan-2-one), poly-para-dioxanone, polyanhydrides such as polymaleic acid anhydrides, polyhydroxy methacrylates, fibrin, polycyanoacrylate, polycaprolactone dimethylacrylates, poly-β-maleic acid, polycaprolactone butyl acrylates, multiblock polymers, e.g. from oligocaprolactonedioles and oligodioxanonedioles, polyetherester multiblock polymers such as PEG and poly(butylene terephthalate), polypivotolactones, polyglycolic acid trimethyl carbonates, polycaprolactone glycolides, poly(γ-ethyl glutamate), poly(DTH-iminocarbonate), poly(DTE-co-DT-carbonate), poly(bisphenol A-iminocarbonate), polyorthoesters, polytrimethyl carbonates, polyiminocarbonates, poly(N-vinyl)-pyrrolidone polyvinyl alcohols, polyester amides, glycolized polyesters, polyphosphoesters, polyphosphazenes, poly[(p-carboxyphenoxy)propane], polyhydroxy pentanoic acid, polyanhydrides, polyethylene oxide propylene oxide, soft polyurethanes, polyurethanes having amino acid residues in the backbone, polyetheresters such as polyethylene oxide, polyalkene oxalates, polyorthoesters as well as copolymers thereof, carrageenans, fibrinogen, starch, collagen, protein based polymers, polyamino acids, synthetic polyamino acids, zein, modified zein, polyhydroxyalkanoates, pectic acid, actinic acid, modified and non modified fibrin and casein, carboxymethyl sulfate, albumin, moreover hyaluronic acid, heparan sulfate, heparin, chondroitin sulfate, dextran, b-cyclodextrins, copolymers with PEG and polypropylene glycol, gum arabic, guar, gelatin, collagen N-hydroxysuccinimide, modifications and copolymers of the aforementioned substances.

In the biodegradable embodiment, such a bioresorbable stent consisting of metal or organic polymers is crimped onto a coated catheter balloon.

The bioresorbable version is advantageous as the stent is completely degraded after a period of from a few weeks to about 18 months and thus no permanent foreign body which could induce chronic inflammations remains in the patient. A sufficient amount of active agent is applied during dilatation by means of the coated catheter balloon, allowing for a controlled ingrowth of the stent which only starts to degrade once ingrowth has been completed and degrades such that no fragments can be washed away via the vessel or respectively the bloodstream.

Furthermore, in said version, the active agent or the combination of active agents can be coated onto the stent surface in form of a layer containing the pure active agent or may be incorporated into a non-polymer matrix, such as a contrast medium, mixture of contrast media or analog of a contrast medium or it may be provided on the stent surface in a polymer carrier, such as one of the biodegradable polymers mentioned above and/or it may be incorporated or introduced into the biodegradable stent material itself.

Consequently this version offers various possibilities for the application or incorporation of one or more active agents onto or into the biodegradable stent. Obviously, it is also possible that one or more active agents are introduced into the biodegradable stent material, i.e. into the stent itself, and that said stent is additionally coated with an active agent or with a polymer or non-polymer carrier containing one or more active agents. Furthermore, the stent or the layer containing the active agent can be provided with a biodegradable barrier layer or a hemocompatible layer, so that systems with two layers or systems with multiple layers are potential embodiments, too.

Additionally, it is also possible that a combination of active agents is incorporated into or applied onto the stent; or a combination of active agents may result from the fact that a different active agent than the one provided on the stent is provided in the stent.

Furthermore, a combination of active agents may also be achieved by the fact that the same active agent is provided on the stent and on the catheter balloon, but in different concentrations, for example in a cytotoxic dose on the catheter balloon and in a cytostatic dose on and/or in the stent.

Preferably, an active agent becoming active within a few hours or days after dilatation is applied onto the catheter balloon, whereas a second active agent is applied onto the stent or incorporated into the biodegradable stent, preferably in a different concentration, which active agent has long-term activity and is released during biodegradation of the stent.

It is particularly preferred that a cytotoxic dose of an active agent is provided on the catheter balloon and that a cytostatic dose of the same or of a different active agent is contained on the stent and/or in the biodegradable stent.

A particularly preferred embodiment contains a cytotoxic dose of paclitaxel on the catheter balloon and in a polymer coating on a metal stent or a cytostatic concentration thereof in a biodegradable coating on the bioresorbable stent.

Another particularly preferred embodiment is represented by a combination of a cytotoxic or a cytostatic dose of paclitaxel on the catheter balloon and a preferably cytostatic dose of rapamycin on or in the biodegradable stent.

Said last mentioned combinations allow for a combination therapy including a rapidly released active agent, preferably in high and/or cytotoxic concentration and a slowly released active agent, preferably in low and/or cytostatic concentration.

The inventive embodiments are suitable for a spontaneous release of a comparatively high amount of active agent, as the interspaces between the struts of the stent and the interspaces between the inner surface of the stent and the surface of the catheter balloon serve as reservoirs for the active agent; i.e. during dilatation, a sufficient amount of active agent from the surface of the catheter balloon is applied in the area between the struts of the stent and preferably in the areas of the vessel wall extending beyond the ends of the stent, in order to successfully prevent an occurrence of restenosis.

Suitable coating solutions for both the catheter balloon and the stent include solutions of e.g. paclitaxel in dimethyl sulfoxide (DMSO) or mixtures of methanol/ethanol or rapamycin in acetic acid ethyl ester or in ethanol. Obviously, other active agents may also be used, in particularly those listed further below.

It is also possible to add a carrier to the solution of active agents, wherein, however, polymer carriers are to be used rather for the stent than for the catheter balloon. If a carrier system is to be used, non-polymer carriers, such as for example contrast media or analogs of contrast media as well as biologically acceptable organic substances, such as amino acids, sugars, vitamins, saccharides and the like are more suited for the coating of the catheter balloon. It is also possible that physiologically acceptable salts are used as matrix for the incorporation of the active agent on the catheter balloon.

Preferably, the balloon is coated on a surface extending beyond the surface covered by the stent. Preferably, the coated area of the balloon extending beyond the end of the stent does not exceed more than 20% of the total length of the stent, preferably no more than 15% and most preferably no more than 10% of the total length of the stent.

Generally a coating of all surfaces of the catheter balloon is advantageous, i.e. all surfaces of the catheter balloon are provided with a coating. The coating of the catheter balloon may further be designed such that the coating with active agent is not uniformly applied, but that instead a gradient is used, i.e. a concentration gradient of the active agent is generated on the balloon surface. Thus, a larger concentration of the active agent may be applied, for example, in the middle of the catheter balloon or at one or both ends or in the middle and at one or both ends of the catheter balloon.

Besides, it is also possible that a higher concentration, with respect to the rest of the surface, of active agent is only applied at one site or on one portion of the catheter balloon. Various variations are possible in this context.

The at least one antiproliferative, antiinflammatory, antiangiogenic, cytostatic, cytotoxic, antithrombotic and/or antirestenotic agent is provided on the catheter balloon, preferably either in form of a layer containing the pure active agent, preferably in dried form, or incorporated into a polymer or non-polymer matrix, wherein the non-polymer matrix is preferred.

Especially contrast media and analogs of contrast media may be used as non-polymer matrix or coating or carrier.

Contrast media and analogs of contrast media are non-polymer compounds which generally have already been clinically approved, are physiologically harmless and can be used when polymer carrier systems and carrier substances are to be avoided.

Analogs of contrast media are contrast-media-like substances having the characteristics of contrast media, i.e. they may be made visible by imaging methods used during surgery.

Contrast media and/or analogs of contrast media contain mostly barium, iodine, manganese, iron, lanthanum, cerium, praseodymium, neodymium, samarium, europium, gadolinium, terbium, dysprosium, holmium, erbium, thulium, ytterbium and/or lutetium, preferably in form of ions in bound and/or complexed form.

Generally, there are different contrast media used for different imaging methods. On the one hand there are contrast media used for x-ray examinations (x-ray contrast media) and on the other hand there are contrast media used for magnetic resonance examinations (MRI contrast media).

X-ray contrast media are substances which either lead to an increased absorption of incident x-rays with respect to the surrounding structure (so called positive contrast media) or which allow for increased smooth penetration of incident x-rays (so called negative contrast media).

Preferred x-ray contrast media include those used for imaging of joints (arthrography) and for CT (computed tomography). A computed tomograph is a device used for producing tomograms of the human body by means of x-rays.

Even though according to the invention x-rays may also be used in imaging methods for detection purposes, said type of radiation is not preferred due to its harmful effects. It is preferred that the incident radiation used is non-ionizing radiation. Used imaging methods include x-ray, computed tomography (CT) and magnetic resonance imaging (MRI), wherein magnetic resonance imaging (MRI) is preferred.

Other preferred contrast media include iodine-containing contrast media which are used for imaging of vessels (angiography and phlebography) and in CT (computed tomography).

Examples for iodine-containing contrast media include the following:

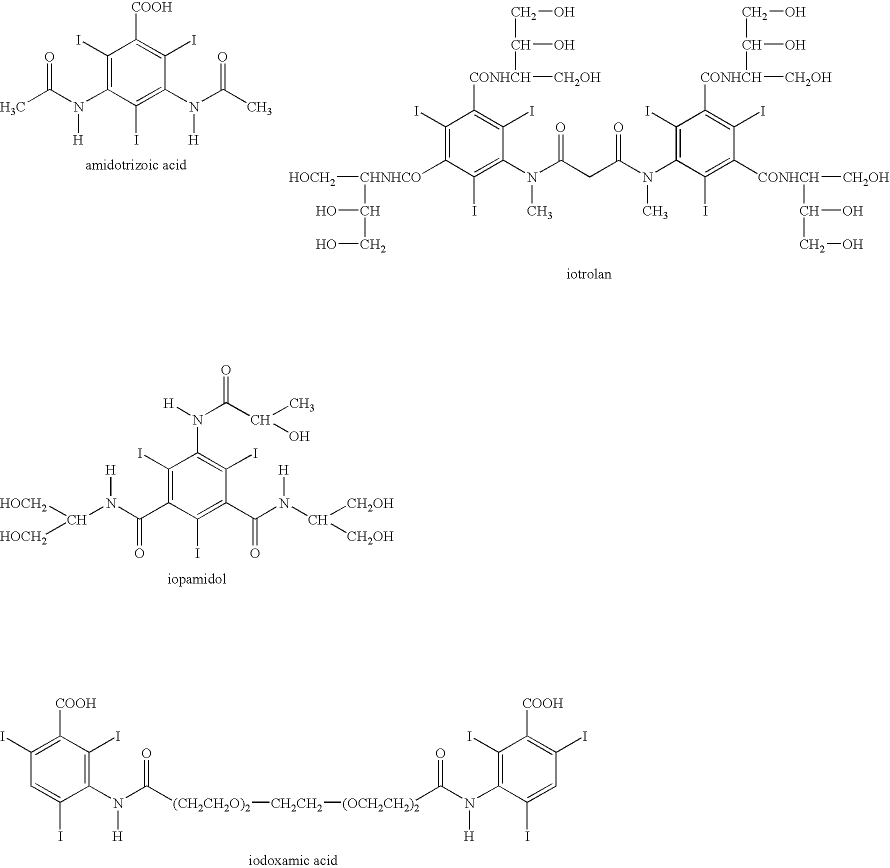

Another example to be mentioned is iodine lipiodol®, an iodized oleum papaveris, a poppy oil. Amidotrizoate, the parent substance of the iodized contrast media is commercially available in form of sodium and meglumine salts under the trade names Gastrograf® in and Gastrolux®.

Gadolunium-containing or superparamagnetic iron particles as well as ferrimagnetic or ferromagnetic iron particles, such as nanoparticles, are preferred.

Another class of preferred contrast media is represented by paramagnetic contrast media, which mostly contain a lanthanoid.

The paramagnetic substances having unpaired electrons include, for example, gadolinium (Gd³⁺) which in total has seven unpaired electrons. Furthermore, said group includes europium (Eu²⁺, Eu³⁺), dysprosium (Dy³⁺) and holmium (Ho³⁺). Said lanthaniods may also be used in chelated form using substances such as hemoglobin, chlorophyll, polyaza acids, polycarboxylic acids and especially EDTA, DTPA, DMSA, DMPS and DOTA as chelating agents.

Examples for contrast media containing gadolinium include gadolinium-diethylenetriaminepentaacetic acid

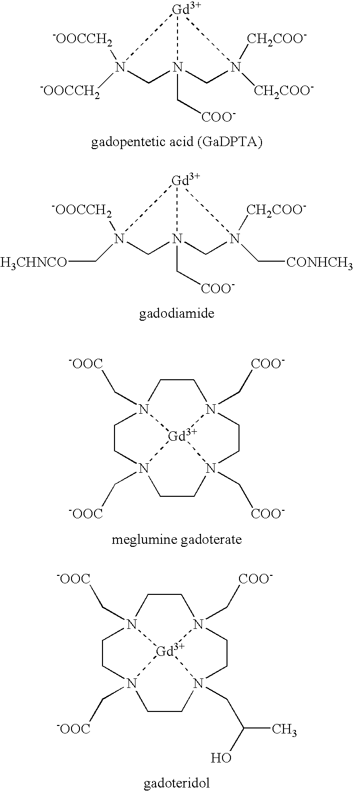

Other paramagnetic substances which may be used according to the invention include ions of so called transition metals such as copper (Cu²⁺), nickel (Ni²⁺), chromium (Cr²⁺, Cr³⁺), manganese (Mn²⁺, Mn³⁺) and iron (Fe²⁺, Fe³⁺). These ions, too, may be used in chelated form.

In addition to the characteristics of the contrast medium or analog of contrast media as carrier or matrix for the active agent, such coatings are further advantageous as visibility, and thus detectability of the catheter balloon in imaging methods is increased.

For example, such contrast media and analogs of contrast media are used for the incorporation of the active agent(s) and especially of paclitaxel or rapamycin. The catheter balloon may be coated with such a mixture or the folds of a catheter balloon may be filled therewith. Additionally, such fluid solution may be released from the inside of the catheter balloon through a plurality of micropores and/or nanopores, preferably under pressure and thus support the detachment of a coating provided on the surface of the balloon. This offers the advantage that a sufficient amount of active agent is supplied to the vessel section during short-time dilatation and the coating is thus uniformly detached from the catheter balloon and pressed against the vessel wall, where it remains and is degraded or respectively resorbed by the cells.

On the other hand, the systems consisting of contrast medium and active agent, especially paclitaxel and rapamycin, are particularly well suited for being incorporated in microrough surfaces or micro cavities, wherein, subsequent to said incorporation, such a coating generally has to be covered with a barrier layer which bursts or is torn open during dilatation but thitherto protects the mixture of contrast medium and active agent from being washed away or degraded to soon.

In order to protect such compositions consisting of contrast medium and active agent from being released too soon, said composition is either applied in or respectively under the folds of multifold balloons or onto the surface of the catheter balloon which may be textured or provided with micro needles or other fillable chambers and which subsequently is preferably covered with a barrier layer. A polymer layer such as disclosed for example in WO 2004/052420 A2 or EP 1 150 622 A1 may be used as barrier layer.

Such barrier layer may consist of polylactides, polyglycolides, polyanhydrides, polyphosphazenes, polyorthoester, polysaccharides, polynucleotides, polypeptides, polyolefins, vinyl chloride polymers, fluoro-containing polymers, teflon, polyvinyl acetates, polyvinyl alcohols, polyvinyl acetals, polyacrylates, polymethacrylates, polystyrene, polyamides, polyimides, polyacetals, polycarbonates, polyesters, polyurethanes, polyisocyanates, polysilicones and copolymers and mixtures of said polymers.

The catheter balloon and the stent are coated in two separate processes, i.e. the catheter balloon is coated without the crimped stent being attached and the stent is coated separately, too.

Possible coating methods include the conventional methods such as spraying, dipping, brushing, plasma deposition and pipetting.

The inventive expandable system consisting of a catheter balloon and a crimped stent may be obtained as follows:
a) providing a balloon for a dilatation catheter;
b) providing a stent;
c) separate coating of the stent and the catheter balloon with an active agent in two different concentrations or with two different active agents;
d) crimping of the coated stent onto the coated catheter balloon.

Thus, catheter balloon and stent are coated separately, wherein it is preferred that a biodegradable coating with one active agent is applied to the stent and that the same or a different active agent is applied to the catheter balloon, preferably in pure form or in a non-polymer carrier system, such as a contrast medium.

The coating of the stent may be additionally provided with a barrier layer which preferably bursts during dilatation, so that the active agent from the layer below is eluted or respectively released.

The stent is only crimped onto the catheter balloon once stent and catheter balloon have been separately coated. Subsequently, they are sterilized and packed.

Therefore, the present invention also relates to expandable systems which can be obtained by such a method.

The active agents from the following list may be selected as antiproliferative, antiinflammatory, antiangiogenic, cytostatic, cytotoxic, antithrombotic and or anti-restenotic agents:

abciximab, acemetacin, acetylvismione B, aclarubicin, ademetionine, adriamycin, aescin, afromosone, akagerine, aldesleukin, amidorone, aminoglutethimide, amsacrine, anakinra, anastrozole, anemonin, anopterine, antimycotics, antithrombotics, apocymarin, argatroban, aristolactam-All, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, combrestatin, Boswellic acids and derivatives thereof, bruceanol A, B and C, bryophyllin A, busulfan, antithrombin, bivalirudin, cadherins, camptothecin, capecitabine, o-carbamoyl-phenoxyacetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type natriuretic peptide (CNP), cudraisoflavone A, curcumin, cyclophosphamide, ciclosporin A, cytarabine, dacarbazine, daclizumab, dactinomycin, dapsone, daunorubicin, diclofenac, 1,11-dimethoxycanthin-6-one, docetaxel, doxorubicin, daunamycin, epirubicin, epothilone A and B, erythromycin, estramustine, etoposide, everolimus, filgrastim, fluroblastin, fluvastatin, fludarabine, fludarabine-5′-dihydrogen phosphate, fluorouracil, folimycin, fosfestrol, gemcitabine, ghalakinoside, ginkgol, ginkgolic acid, glycoside 1a, 4-hydroxyoxycyclophosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, inhibitor 2ω of smc proliferation, mitoxantrone, mycophenolate mofetil, c-myc antisense, β-myc antisense, β-lapachone, podophyllotoxin, podophyllic acid 2-ethyl hydrazide, molgramostim (rhuGM-CSF), peginterferon α-2b, lenograstim (r-HuG-CSF), macrogol, selectin (cytokine antagonist), cytokinin inhibitors, COX-2 inhibitor, NFkB, angiopeptin, monoclonal antibodies inhibiting muscle cell proliferation, bFGF antagonists, probucol, prostaglandins, 1-hydroxy-11-methoxycanthin-6-one, scopoletin, NO donors such as pentaerythritol tetranitrate and sydnonimines, S-nitroso derivatives, tamoxifen, staurosporine, β-estradiol, α-estradiol, estriol, estrone, ethinyl estradiol, medroxyprogesterone, estradiol cypionates, estradiol benzoates, tranilast, kamebakaurin and other terpenoids used in cancer therapy, verapamil, tyrosine kinase inhibitors (tyrphostins), paclitaxel and derivatives thereof such as 6-α-hydroxy-paclitaxel, taxotere, carbon suboxide (MCS) and macrocyclic oligomers thereof, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtecaine, polidocanol, nonivamide, levomenthol, ellipticine, D-24851 (Calbiochem), colcemid, cytochalasin A-E, indanocine, nocodazole, S100 protein, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor 1, plasminogen activator inhibitor 2, antisense oligonucleotides, VEGF inhibitors, IGF1, active agents from the group of antibiotics such as cefadroxil, cefazolin, cefaclor, cefoxitin, tobramycin, gentamicin, penicillins such as dicloxacillin, oxacillin, sulfonamides, metronidazole, enoxaparin, desulfated and N-reacetylated heparin (hemoparin®), tissue plasminogen activator, GpIIb/IIIa platelet membrane receptor, antibodies to factor Xₐ inhibitor, heparin, hirudin, rhirudin, PPACK, protamine, prourokinase, streptokinase, warfarin, urokinase, vasodilators such as dipyramidole, trapidil, nitroprussides, PDGF antagonists such as triazolopyrimidine and seramin, ACE inhibitors such as captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators such as p65, NF-kB or Bcl-xL antisense oligonucleotides, halofuginone, nifedipine, tocopherol, molsidomine, tea polyphenols, epicatechin gallate, epigallocatechin gallate, leflunomide, etanercept, sulfasalazine, tetracycline, triamcinolone, mutamycin, procainimide, retinoic acid, quinidine, disopyrimide, flecainide, propafenone, sotalol, natural and synthetically obtained steroids such as inotodiol, maquiroside A, ghalakinoside, mansonine, strebloside, hydrocortisone, betamethasone, dexamethasone, non-steroidal substances (NSAIDS) such as fenoprofen, ibuprofen, indomethacin, naproxen, phenylbutazone and other antiviral agents such as acyclovir, ganciclovir and zidovudine, clotrimazole, flucytosine, griseofulvin, ketoconazole, miconazole, nystatin, terbinafine, antiprotozoal agents such as chloroquine, mefloquine, quinine, moreover natural terpenoids such as hippocaesculin, barringtogenol-C21-angelate, 14-dehydroagrostistachin, agroskerin, agrostistachin, 17-hydroxyagrostistachin, ovatodiolids, 4,7-oxycycloanisomelic acid baccharinoids B1, B2, B3 and B7, tubeimoside, bruceantinoside C, yadanziosides N and P, isodeoxyelephantopin, tomenphantopin A and B, coronarin A, B, C and D, ursolic acid, hyptatic acid A, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrin, taxamairin A and B, regenilol, triptolide, moreover cymarin, hydroxyanopterine, protoanemonin, cheliburin chloride, sinococuline A and B, dihydronitidine, nitidine chloride, 12-6-hydroxypregnadien-3,20-dione, helenalin, indicine, indicine-N-oxide, lasiocarpine, inotodiol, justicidin A and B, larreatin, malloterin, mallotochromanol, isobutyrylmallotochromanol, marchantin A, maytansin, lycoridicin, margetine, pancratistatin, liriodenine, oxoushinsunine, periplocoside A, deoxypsorospermin, psychorubin, ricin A, sanguinarine, manwu wheat acid, methylsorbifolin, chromones of spathelia, stizophyllin, dihydrousambaraensine, hydroxyusambarine, strychnopentamine, strychnophylline, usambarine, usambarensine, liriodenine, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B, zeorin.

As already mentioned before, the active agent may also be applied in pure form or together with a polymer or a non-polymeric carrier. Dipping or coating methods are preferably used for said application.

Furthermore, the antiproliferative, antiinflammatory, antiangiogenic, cytostatic, cytotoxic, antithrombotic and/or anti-restenotic agent can be incorported into a polymer matrix or may be provided underneath and/or on polymer matrix.

Besides, an additional, polymeric biostable or biodegradable layer can be additionally provided on the stent. Said layer may equally contain another antiproliferative, antiinflammatory, antiangiogenic, cytostatic, cytotoxic, antithrombotic and or anti-restenotic agent which may be identical or different to the active agent in the polymer matrix.

The following substance may be used as biostable or biodegradable polymer and/or polymers for the polymer matrix:

polyverolactone, poly-ε-decalactone, polylactonic acid, polyglycolic acid, polylactides, polyglycolides, copolymers of the polylactides and polyhydroxyvalerates, ε-carprolactone, polyhydroxybutyric acid, polyhydroxybutyrates, polyhydroxyvalerates, polyhydroxybutyrate-co-valerate, poly(1,4-dioxan-2,3-one), poly(1,3-dioxan-2-one), poly-para-dioxanone, polyanhydrides, polymaleic acid, anhydride, polyhydroxy methacrylates, fibrin, polycyanoacrylate, polycaprolactone dimethylacrylates, poly-β-maleic acid, polycaprolactone butyl acrylate, multiblock polymers from oligocaprolactonedios and oligodioxanonediols, polyether ester multiblock polymers from PEG and poly(butylene terephthalate), polypivotolactones, polyglycolic acid trimethyl carbonates, polycaprolactone glycolides, poly(γ-ethyl glutamate), poly(DTH-iminocarbonate), poly(DTE-co-DT-carbonate), poly(bisphenol A-iminocarbonate). polyorthoester, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-carboxyphenoxy)propane], polyhydroxy pentanoic acid, polyanhydrides, polyethylene oxide propylene oxide, soft polyurethanes, polyurethanes having amino acid residues in the backbone, polyetheresters such as polyethylene oxide, polyalkene oxalates, polyorthoesters as well as copolymers thereof, lipids, carrageenanes, fibrinogen, starch, collagen, protein based polymers, polyamino acids, synthetic polyamino acids, zein, polyhydroxyalkanoates, pectic acid, actinic acid, carboxymethyl sulfate, albumin, hyaluronic acid, chitosan and derivatives thereof, heparan sulfates and derivates thereof, heparins, chondroitin sulfate, dextran, β-cyclodextrins, copolymers with PEG and polypropylene glycol, gum arabic, guar, gelatin, collagen, N-hydroxysuccinimide, phospholipids, polyacrylic acid, polyacrylates, polymethyl methacrylate, polybutyl methacrylate, polyacrylamide, polyacrylonitriles, polyamides, polyetheramides, polyethylene amine, polyimides, polycarbonates, polycarbourethanes, polyvinyl ketanoes, polyvinyl halogenides, polyvinylidene halogenides, polyvinyl ethers, polyisobutylenes, polyvinyl aromatics, polyvinyl esters, polyvinyl pyrrolidones, polyoxymethylenes, polytetramethylene oxide, polyethylene, polypropylene, polytetrafluoroethylene, polyurethanes, polyether urethanes, silicone polyether urethanes, silicone polyurethanes, silicone polycarbonate urethanes, polyolefin elastomers, EPDM gums, fluorosilicones, carboxymethyl chitosans polyaryletheretherketones, polyetheretherketones, polyethylene terephthalate, polyvalerates, carboxymethylcellulose, cellulose, rayon, rayon triacetates, cellulose nitrates, cellulose acetates, hydroxyethyl cellulose, cellulose butyrates, cellulose acetate butyrates, ethyl vinyl acetate copolymers, polysulfones, epoxy resins, ABS resins, silicones such as polysiloxanes, polydimethylsiloxanes, polyvinyl halogens and copolymers, cellulose ethers, cellulose triacetates, chitosans and copolymers and/or mixtures of the aforementioned polymers.

The inventive expandable systems are perfectly suited for the prophylaxis, prevention or reduction of restenosis.

### EXAMPLES

### Example 1

A commercially available catheter with a catheter balloon made of polyamide is coated with a solution of paclitaxel in DMSO by means of a spraying method.

The coating is dried after each spraying round and the spray coating process is repeated three times.

An amorphous, uniform coating of the whole surface of the catheter balloon is obtained.

A commercially available cobalt-chromium stent is provided with a carbon layer and subsequently coated with a polymer coating consisting of a polylactide-polyglycolide. The polymeric, biodegradable coating contains the active agent rapamycin, preferably in a cytostatic concentration.

The coated stent is then crimped onto the coated catheter balloon.

### Example 2

A commercially available catheter with a catheter balloon made of polyamide is coated with a preferably cytotoxic solution of paclitaxel in DMSO by means of a dipping method.

The coating is dried after each dipping process and the dipping process is repeated two times.

An amorphous, uniform coating of the whole surface, including the folds of the catheter balloon, is obtained.

A commercially available vanadium stent is coated with a polymer coating consisting of a polyurethane containing paclitaxel, preferably in a cytostatic concentration.

The coated stent is then crimped onto the coated catheter balloon.

1. Expandable system consisting of a catheter balloon and a crimped stent, wherein the balloon and the stent release one or different active agents with different release kinetics.

2. Expandable system according to claim 1, wherein the system is suitable for the targeted prophylaxis and/or treatment of restenosis due to a combination of two active agents having different release kinetics or to one active agent in different concentrations and having different release kinetics.

3. Expandable system according to claim 1, wherein the catheter balloon is capable of releasing an active agent having a rapid release rate and wherein the stent is capable of releasing an active agent having a slow release rate.

4. Expandable system according to claim 1, wherein an antiproliferative, antiinflammatory, antiangiogenic, cytostatic, cytotoxic, antithrombotic, and/or anti-restenotic agent is provided on the catheter balloon.

5. Expandable system according to claim 1, wherein an antiproliferative, antiinflammatory, antiangiogenic, cytostatic, cytotoxic, antithrombotic and/or anti-restenotic agent is provided on the stent.

6. Expandable system according to claim 4, wherein a cytotoxic amount of an antiproliferative, antiinflammatory, antiangiogenic, cytostatic, cytotoxic, antithrombotic and/or anti-restenotic agent is provided on the catheter balloon.

7. Expandable system according to claim 5, wherein a cytostatic amount of an antiproliferative, antiinflammatory, antiangiogenic, cytostatic, cytotoxic, antithrombotic and/or anti-restenotic agent is provided on the stent.

8. Expandable system according to claim 1, wherein a ten times higher amount, with respect to the stent, of an antiproliferative, antiinflammatory, antiangiogenic, cytostatic, cytotoxic, antithrombotic, antiinflammatory and/or anti-restenotic agent is provided on the catheter balloon.

9. Expandable system according to claim 1, wherein the stent is bioresorbable.

10. Expandable system according to claim 1, wherein the antiproliferative, antiinflammatory, antiangiogenic, cytostatic, cytotoxic, antithrombotic and/or anti-restenotic agent is selected from the group comprising:
abciximab, acemetacin, acetylvismione B, aclarubicin, ademetionine, adriamycin, aescin, afromosone, akagerine, aldesleukin, amidorone, aminoglutethimide, amsacrine, anakinra, anastrozole, anemonin, anopterine, antimycotics, antithrombotics, apocymarin, argatroban, aristolactam-AII, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, combrestatin, Boswellic acids and derivatives thereof, bruceanol A, B and C, bryophyllin A, busulfan, antithrombin, bivalirudin, cadherins, camptothecin, capecitabine, o-carbamoyl-phenoxyacetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type natriuretic peptide (CNP), cudraisoflavone A, curcumin, cyclophosphamide, ciclosporin A, cytarabine, dacarbazine, daclizumab, dactinomycin, dapsone, daunorubicin, diclofenac, 1,11-dimethoxycanthin-6-one, docetaxel, doxorubicin, daunamycin, epirubicin, epothilone A and B, erythromycin, estramustine, etoposide, everolimus, filgrastim, fluroblastin, fluvastatin, fludarabine, fludarabine-5′-hydrogen phosphate, fluorouracil, folimycin, fosfestrol, gemcitabine, ghalakinoside, ginkgol, ginkgolic acid, glycoside 1a, 4-hydroxyoxycyclophosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, inhibitor 2ω of smc proliferation, mycophenolate mofetil, c-myc antisense, β-myc antisense, β-lapachone, podophyllotoxin, podophyllic acid 2-ethyl hydrazide, molgramostim (rhuGM-CSF), peginterferon α-2b, lenograstim (r-HuG-CSF), macrogol, selectin (cytokine antagonist), cytokinin inhibitors, COX-2 inhibitor, NFkB, angiopeptin, monoclonal antibodies inhibiting muscle cell proliferation, bFGF antagonists, probucol, prostaglandins, 1-hydroxy-11-methoxycanthin-6-one, scopoletin, NO donors such as pentaerythritol tetranitrate and sydnonimines, S-nitroso derivatives, tamoxifen, staurosporine, 13-estradiol, α-estradiol, estriol, estrone, ethinyl estradiol, medroxyprogesterone, estradiol cypionates, estradiol benzoates, tranilast, kamebakaurin and other terpenoids used in cancer therapy, verapamil, tyrosine kinase inhibitors (tyrphostins), paclitaxel and derivatives thereof such as 6-α-hydroxy-paclitaxel, taxotere, carbon suboxide (MCS) and macrocyclic oligomers thereof, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtecaine, polidocanol, nonivamide, levomenthol, ellipticine, D-24851 (Calbiochem), colcemid, cytochalasin A-E, indanocine, nocodazole, S100 protein, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor-1, plasminogen activator inhibitor-2, antisense oligonucleotides, VEGF inhibitors, IGF1, active agents from the group of antibiotics such as cefadroxil, cefazolin, cefaclor, cefoxitin, tobramycin, gentamicin, penicillins such as dicloxacillin, oxacillin, sulfonamides, metronidazole, enoxaparin, desulfated and N-reacetylated heparin (Hemoparin®), tissue plasminogen activator, GpIIb/IIIa platelet membrane receptor, antibodies to factor Xₐ inhibitor, heparin, hirudin, r-hirudin, PPACK, protamine, prourokinase, streptokinase, warfarin, urokinase, vasodilators such as dipyramidole, trapidil, nitroprussides, PDGF antagonists such as triazolopyrimidine and seramin, ACE inhibitors such as captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators such as p65, NF-kB or Bcl-xL antisense oligonucleotides, halofuginone, nifedipine, tocopherol, molsidomine, tea polyphenols, epicatechin gallate, epigallocatechin gallate, leflunomide, etanercept, sulfasalazine, dicloxacillin, tetracycline, triamcinolone, mutamycin, procainimide, retinoic acid, quinidine, disopyrimide, flecainide, propafenone, sotalol, natural and synthetically obtained steroids such as inotodiol, maquiroside A, ghalakinoside, mansonine, strebloside, hydrocortisone, betamethasone, dexamethasone, non-steroidal substances (NSAIDS) such as fenoprofen, ibuprofen, indomethacin, naproxen, phenylbutazone and other antiviral agents such as acyclovir, ganciclovir and zidovudine, clotrimazole, flucytosine, griseofulvin, ketoconazole, miconazole, nystatin, terbinafine, antiprotozoal agents such as chloroquine, mefloquine, quinine, moreover natural terpenoids such as hippocaesculin, barringtogenol-C21-angelate, 14-dehydroagrostistachin, agroskerin, agrostistachin, 17-hydroxyagrostistachin, ovatodiolids, 4,7-oxycycloanisomelic acid, baccharinoids B1, B2, B3 and B7, tubeimoside, bruceantinoside C, yadanziosides N and P, isodeoxyelephantopin, tomenphantopin A and B, coronarin A, B, C and D, ursolic acid, hyptatic acid A, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrin, taxamairin A and B, regenilol, triptolide, moreover cymarin, hydroxyanopterine, protoanemonin, cheliburin chloride, sinococuline A and B, dihydronitidine, nitidine chloride, 12-β-hydroxypregnadien-3,20-dione, helenalin, indicine, indicine-N-oxide, lasiocarpine, inotodiol, justicidin A and B, larreatin, malloterin, mallotochromanol, isobutyrylmallotochromanol, maquiroside A, marchantin A, maytansine, lycoridicin, margetine, pancratistatin, liriodenine, bisparthenolidin, oxoushinsunine, periplocoside A, ursolic acid, deoxypsorospermin, psychorubin, ricin A, sanguinarine, manwu wheat acid, methylsorbifolin, chromones of spathelia, stizophyllin, dihydrousambaraensine, hydroxyusambarine, strychnopentamine, strychnophylline, usambarine, usambarensine, liriodenine, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B, zeorin.

11. Expandable system according to claim 1, wherein an antiproliferative, antiinflammatory, antiangiogenic, cytostatic, cytotoxic, antithrombotic and/or anti-restenotic agent is provided in and/or on and/or underneath a polymer matrix.

12. Expandable system according to claim 11, wherein the polymer(s) for the polymer matrix are selected from the group comprising:
polyvalerolactone, poly-ε-decalactone, polylactonic acid, polyglycolic acid, polylactides, polyglycolides, copolymers of the polylactides and polyglycolides, poly-ε-caprolactone, polyhydroxybutyric acid, polyhydroxybutyrates, polyhydroxyvalerates, polyhydroxybutyrate-co-valerate, poly(1,4-dioxan-2,3-one), poly(1,3-dioxan-2-one), poly-para-dioxanone, polyanhydrides, polymaleic acid anhydride, polyhydroxymethacrylates, fibrin, polycyanoacrylate, polycaprolactone dimethylacrylates, poly-β-maleic acid, polycaprolactone butyl acrylate, multiblock polymers from oligocaprolactonediols and oligodioxanonediols, polyether ester multiblock polymers from PEG and poly(butylene terephthalates), polypivotolactones, polyglycolic acid trimethyl carbonates, polycaprolactone glycolides, poly(γ-ethyl glutamate) poly(DTH-iminocarbonate), poly(DTE-co-DT-carbonate), poly(bisphenol A-iminocarbonate), polyorthoesters, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-carboxyphenoxy)propane], polyhydroxy pentanoic acid, polyanhydrides, polyethylene oxide propylene oxide, soft polyurethanes, polyurethanes having amino acid residues in the backbone, polyetheresters such as polyethylene oxide, polyalkene oxalates, polyorthoesters as well as copolymers thereof, lipids, carrageenans, fibrinogen, starch, collagen, protein based polymers, polyamino acids, synthetic polyamino acids, zein, polyhydroxyalkanoates, pectic acid, actinic acid, carboxymethyl sulfate, albumin, hyaluronic acid, chitosan and derivatives thereof, heparan sulfates and derivates thereof, heparins, chondroitin sulfate, dextran, β-cyclodextrins, copolymers with PEG and polypropylene glycol, gum arabic, guar, gelatin, collagen N-hydroxysuccinimide, phospholipids, polyacrylic acid, polyacrylates, polymethyl methacrylate, polybutyl methacrylate, polyacrylamide, polyacrylonitriles, polyamides, polyetheramides, polyethylene amine, polyimides, polycarbonates, polycarbourethanes, polyvinyl ketones, polyvinyl halogenides, polyvinylidene halogenides, polyvinyl ethers, polyisobutylenes, polyvinyl aromatics, polyvinyl esters, polyvinyl pyrrolidones, polyoxymethylenes, polytetramethylene oxide, polyethylene, polypropylene, polytetrafluoroethylene, polyurethanes, polyether urethanes, silicone polyether urethanes, silicone polyurethanes, silicone polycarbonate urethanes, polyolefin elastomers, EPDM gums, fluorosilicones, carboxymethyl chitosans, polyaryletheretherketones, polyetheretherketones, polyethylene terephthalate, polyvalerates, carboxymethylcellulose, cellulose, rayon, rayon triacetates, cellulose nitrates, cellulose acetates, hydroxyethyl cellulose, cellulose butyrates, cellulose acetate butyrates, ethyl vinyl acetate copolymers, polysulfones, epoxy resins, ABS resins, silicones such as polysiloxanes, polydimethylsiloxanes, polyvinyl halogens and copolymers, cellulose ethers, cellulose triacetates, chitosans and copolymers and/ or mixtures of the aforementioned polymers.

13. Expandable system according to claim 1, wherein an additional biostable or biodegradable layer is provided underneath and/or on the layer containing an antiproliferative, antiinflammatory, antiangiogenic, cytostatic, cytotoxic, antithrombotic and/or anti-restenotic agent.

14. Method for coating an expandable system consisting of a catheter balloon and a crimped stent comprising the following steps:
a) providing a balloon for a dilatation catheter;
b) providing a stent;
c) separate coating of the stent and the catheter balloon with an active agent in two different concentrations or with two different active agents;
d) crimping of the coated stent on the coated catheter balloon.

15. Coating method according to claim 14, further comprising step
c′) application onto the stent of a biostable and/or biodegradable layer as exterior layer.

16. Expandable system which can be obtained by a method according to claim 14.

17. Expandable system with attached stent according to claim 1 to be used for the prophylaxis, prevention or reduction of restenosis.
